(19) United States
(12) Patent Application Publication
(10) Pub. No.: US 2008/0171909 A1
(43) Pub. Date: Jul. 17, 2008

(54) ENDOSCOPE AND ENDOSCOPIC SYSTEM

(75) Inventors: Fumiyuki Onoda, Tokyo (JP); Sumihiro Uchimura, Sagamihara-shi (JP); Toshiaki Noguchi, Tokyo (JP); Katsuya Suzuki, Sagamihara-shi (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS CORPORATION, Tokyo (JP)

(21) Appl. No.: 12/051,217

(22) Filed: Mar. 19, 2008

Related U.S. Application Data
(62) Division of application No. 11/509,504, filed on Aug. 24, 2006.
Application No. 11/509,504 is a continuation of application No. PCT/JP2005/003326, filed on Feb. 28, 2005.

(30) Foreign Application Priority Data
Feb. 27, 2004 (JP) 2004-054675
Feb. 27, 2004 (JP) 2004-054676

(51) Int. Cl. A61B 1/00

(52) U.S. Cl. 600/118; 600/101

(57) ABSTRACT

An endoscope of the present invention includes an insert section inserted in a subject body, an operation section provided on a base end side of the insert section, a grasping section provided to the operation section and formed to be substantially bilaterally symmetric to a reference line extending in a longitudinal direction, and a plurality of instruction input sections provided to be substantially bilaterally symmetric to the reference line extending in the longitudinal direction of the grasping section.

### CROSS REFERENCES TO RELATED APPLICATIONS

This application is a continuation application of PCT/JP2005/003326 filed on Feb. 28, 2005 and claims benefit of Japanese Applications No. 2004-054675 filed in Japan on Feb. 27, 2004 and No. 2004-054676 filed in Japan on Feb. 27, 2004, the entire contents of each of which are incorporated herein by their reference.

### BACKGROUND OF THE INVENTION

1. Field of the Invention

The present invention relates to an endoscope to be inserted in a body cavity or the like for performing an endoscopy or the like and an endoscopic system having the endoscope.

2. Description of the Related Art

In recent years, endoscopes having an illumination section and an observation section at a distal end of an elongated insert section are being widely adopted in a medical field and an industrial field.

For example, an endoscope is known which has a flexible insert section and a bending section in the vicinity of a distal end of the insert section for allowing an observation in a desired direction after being inserted in a winding body, or the like. Also, as an endoscope of this type, there is known one enabling a bending operation (articulation operation) for bending a bending section by using an operation section at hand.

In addition, an endoscope having various switches at an operation section is known. For example, an endoscope having a freeze switch or the like for instructing a still-picture display is known in an endoscope having a signal processing device for performing a signal processing on an image pickup element built in a distal end section. It should be noted that in the endoscope of this type, in general, a surgeon performs various operations by a hand which grasps a grasping section of the operation section.

Furthermore, as an endoscope of this type, for example, Japanese Unexamined Patent Application Publication No. 2002-58629 discloses an electronic endoscope. The endoscope has bending operation knobs, suction buttons, and air water buttons on two adjacent side sections which form a grasping section to be grasped by a surgeon.

In such an endoscope, for example, when the grasping section is grasped by a left hand, the bending operation knob is arranged at a side position where the thumb of the left hand can reach, and the suction button and the air water button are arranged at positions where the index finger and the middle finger can instruct the operations of suction, and airing and watering.

Thus, the surgeon performs the articulation operation by the thumb of the grasping left hand when the endoscope is grasped by the left hand, and suction, and airing and watering can be performed by the index finger and the middle finger.

In the endoscope described in the above-mentioned publication, if the surgeon grasps the grasping section with a right hand, the operation is extremely difficult. For this reason, in this endoscope, if the arrangement positions of the bending operation knob, the suction button, and the air water button are not changed for the surgeon who grasps the endoscope with the right hand, there is a problem in that satisfactory operability cannot be ensured.

In this manner, some endoscopes have a problem in that operability is degraded depending on the left and or the right hand of the surgeon for grasping the grasping section.

Meanwhile, Japanese Unexamined Patent Application Publication No. 2002-58629 discloses an endoscope having an instruction input section and the like such as a bending operation knob on a rear side of a grasping section formed near an insert section, in which a universal cable is extended from a side further back of the grasping section.

In the endoscope of the previous cases, the endoscope of this type may degrade the operability due to the existence of the universal cable when a predetermined operation is performed. For this reason, the endoscope disclosed in the publication includes the universal cable in the insert section side rather than the grasping section side. Also, in the electronic endoscope, a canopy section is provided between the suction button and the air water button in the grasping section, and thus, when the grasping section is grasped, discrimination between the suction button and the air water button becomes facilitated by the sense of the fingers.

Moreover, in the endoscope described in the publication, for example, when the grasping section is grasped by the left hand, the bending operation knob is arranged at a side position where the thumb of the left hand can reach, and also the suction button and the air water button are arranged in such a manner that the index finger and the middle finger can instruct the suction, and airing and watering. Therefore, when the surgeon grasps the grasping section, for example, by the left hand, the thumb of the grasping left hand can perform the articulation operation, and the index finger and the middle finger can instruct the operations of suction, and airing and watering.

However, in the electronic endoscope described in the above-mentioned publication, when the surgeon is tired from using the hand grasping the grasping section, and intends to release the hand grasping the grasping section, only a small canopy section is provided on the side face of the grasping section, so a sufficient release status cannot be achieved for preventing drop of the electronic endoscope.

Also, in the electronic endoscope described in the above-mentioned publication, when various operations are performed while grasping the grasping section, the drop of the electronic endoscope cannot be prevented unless the grasping section is firmly held, and an accurate operation is difficult to perform.

### SUMMARY OF THE INVENTION

The present invention has been made in view of the above-mentioned problems and it is therefore an object to provide an endoscope with which a satisfactory operability can be ensured without firmly grasping a grasping section even when a left hand or a right hand grasps the grasping section.

The endoscope according to an aspect of the present invention includes an insert section inserted in a subject body; an operation section provided on a base end side of the insert section; a grasping section provided to the operation section and formed to be substantially bilaterally symmetric to a reference line extending in a longitudinal direction; and a plurality of instruction input sections provided to be substantially bilaterally symmetric to the reference line extending in the longitudinal direction of the grasping section.

### BRIEF DESCRIPTION OF THE DRAWINGS

FIG. 1 shows a schematic structure of an endoscopic system according to a first embodiment of the present invention.

FIG. 2A is a block diagram showing an example of data communication mode in the endoscopic system according to the first embodiment.

FIG. 2B is a block diagram showing an example of data communication mode in the endoscopic system according to the first embodiment.

FIG. 2C is a block diagram showing an example of data communication mode in the endoscopic system according to the first embodiment.

FIG. 3 is a perspective view showing a specific outer appearance shape of an AWS unit peripheral section in the endoscopic system according to the first embodiment.

FIG. 4A is a perspective view showing a state in which a detachable AWS adapter is attached to the AWS unit in the endoscopic system according to the first embodiment.

FIG. 4B is a perspective view showing a state in which the detachable AWS adapter is removed from the AWS unit in the endoscopic system according to the first embodiment.

FIG. 5A is a front view showing a structure of the AWS adapter in the endoscopic system according to the first embodiment.

FIG. 5B is a side view showing the structure of the AWS adapter in the endoscopic system according to the first embodiment.

FIG. 5C is another side view showing the structure of the AWS adapter in the endoscopic system according to the first embodiment.

FIG. 5D is a cross-sectional view showing the structure of the AWS adapter in the endoscopic system according to the first embodiment.

FIG. 5E is another cross-sectional view showing the structure of the AWS adapter in the endoscopic system according to the first embodiment.

FIG. 6 shows internal structures of a control device and the AWS unit and a structure of a connection section of an endoscope connector in the endoscopic system according to the first embodiment.

FIG. 7 is a side view with a see-through of a part of an internal structure component of an endoscope in the endoscopic system according to the first embodiment.

FIG. 8A is a side view showing a specific outer appearance shape of the endoscope in the endoscopic system according to the first embodiment.

FIG. 8B is a front view showing the specific outer appearance shape of the endoscope in the endoscopic system according to the first embodiment.

FIG. 8C is a back view showing the specific outer appearance shape of the endoscope in the endoscopic system according to the first embodiment.

FIG. 8D is a plan view showing the specific outer appearance shape of the endoscope as seen from the front in the endoscopic system according to the first embodiment.

FIG. 8E is a main part enlarged view showing an example of a nearly optimal angle range of an inclined surface in the endoscope in the endoscopic system according to the first embodiment.

FIG. 9A shows a structure of a transparency sensor of the endoscope in the endoscopic system according to the first embodiment.

FIG. 9B shows an operation of a transparency sensor of the endoscope in the endoscopic system according to the first embodiment.

FIG. 10 is a block diagram showing an electric structure of the endoscope in the endoscopic system according to the first embodiment.

FIG. 11A shows a representative display example of a monitor display screen of an observation monitor in the endoscopic system according to the first embodiment.

FIG. 11B shows a specific display example of a menu display of the monitor display screen of the observation monitor in the endoscopic system according to the first embodiment.

FIG. 11C shows another specific display example of a menu display of the monitor display screen of the observation monitor in the endoscopic system according to the first embodiment.

FIG. 12A is a perspective view showing a state in which an electromagnetic unit that is a modified example of the AWS adapter is attached to the AWS unit in the endoscopic system according to the first embodiment.

FIG. 12B is a perspective view showing a state in which the electromagnetic unit that is a modified example of the AWS adapter is removed the AWS unit in the endoscopic system according to the first embodiment.

FIG. 13A is a front view showing a structure of a modified example of the AWS adapter in the endoscopic system according to the first embodiment.

FIG. 13B is a side view showing the structure of the modified example of the AWS adapter in the endoscopic system according to the first embodiment.

FIG. 13C is another side view showing the structure of the modified example of the AWS adapter in the endoscopic system according to the first embodiment.

FIG. 13D is a cross-sectional view showing the structure of the modified example of the AWS adapter in the endoscopic system according to the first embodiment.

FIG. 13E is another cross-sectional view showing the structure of the modified example of the AWS adapter in the endoscopic system according to the first embodiment.

FIG. 14A is a side view showing a specific outer appearance shape of the endoscope in the endoscopic system according to a second embodiment.

FIG. 14B is a front view showing the specific outer appearance shape of the endoscope in the endoscopic system according to the second embodiment.

FIG. 14C is a plan view showing the specific outer appearance shape of the endoscope as seen from the front in the endoscopic system according to the second embodiment.

FIG. 14D shows a specific outer appearance shape of a first modified example of the endoscope in the endoscopic system according to the second embodiment of the present invention.

FIG. 15A is a side view showing an operation section of a second modified example of the endoscope in the endoscopic system according to the second embodiment of the present invention.

FIG. 15B is a front view showing the operation section of the second modified example of the endoscope in the endoscopic system according to the second embodiment of the present invention.

FIG. 15C is a plan view showing the operation section of the second modified example of the endoscope as seen from the top in the endoscopic system according to the second embodiment of the present invention.

FIG. 15D shows an example of an operation pad in an arrangement state along a center line parallel to an inclination surface Sa as seen from a direction perpendicular to the inclination surface Sa in FIG. 15A.

FIG. 15E shows another example of the operation pad in the arrangement state along the center line parallel to the inclination surface Sa as seen from the direction perpendicular to the inclination surface Sa in FIG. 15A.

FIG. 16A is a side view showing an operation section of a third modified example of the endoscope in the endoscopic system according to the second embodiment of the present invention.

FIG. 16B is a front view showing the operation section of the third modified example of the endoscope in the endoscopic system according to the second embodiment of the present invention.

FIG. 16C is a plan view showing the operation section of the third modified example of the endoscope as seen from the top in the endoscopic system according to the second embodiment of the present invention.

FIG. 16D shows an example of an operation pad in an arrangement state along a center line parallel to an inclination surface Sa as seen from a direction perpendicular to the inclination surface Sa in FIG. 16A.

FIG. 17A is a side view showing an operation section of a fourth modified example of the endoscope in the endoscopic system according to the second embodiment of the present invention.

FIG. 17B is a front view showing the operation section of the fourth modified example of the endoscope in the endoscopic system according to the second embodiment of the present invention.

FIG. 17C is a plan view showing the operation section of the fourth modified example of the endoscope as seen from the top in the endoscopic system according to the second embodiment of the present invention.

FIG. 17D shows an example of an operation pad in an arrangement state along a center line parallel to an inclination surface Sa as seen from a direction perpendicular to the inclination surface Sa in FIG. 17A.

FIG. 18 shows structures of an operation section and a peripheral section thereof of a fifth modified example of the endoscope in the endoscopic system according to the second embodiment of the present invention.

### DESCRIPTION OF THE PREFERRED EMBODIMENTS

Preferred embodiments of the present invention will be described below with reference to the drawings.

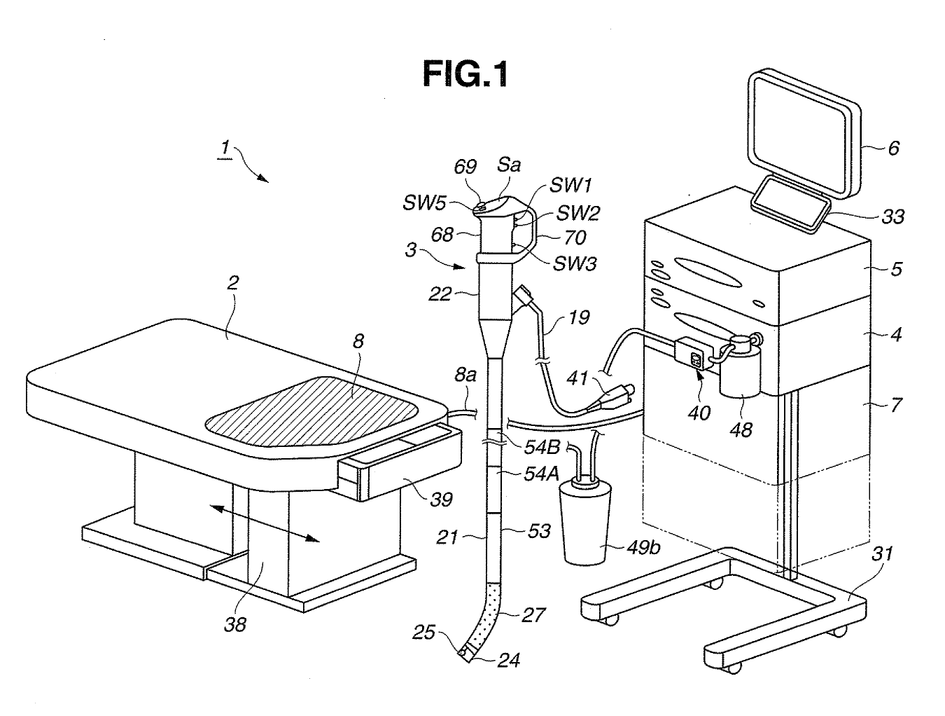

Before a specific structure of an endoscopic system according to a first embodiment of the present invention will be described, a description is first given of a schematic structure of the endoscopic system with reference to FIGS. 1 to 3.

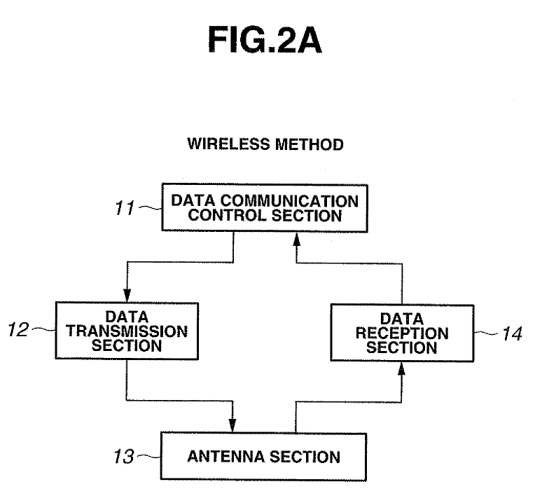

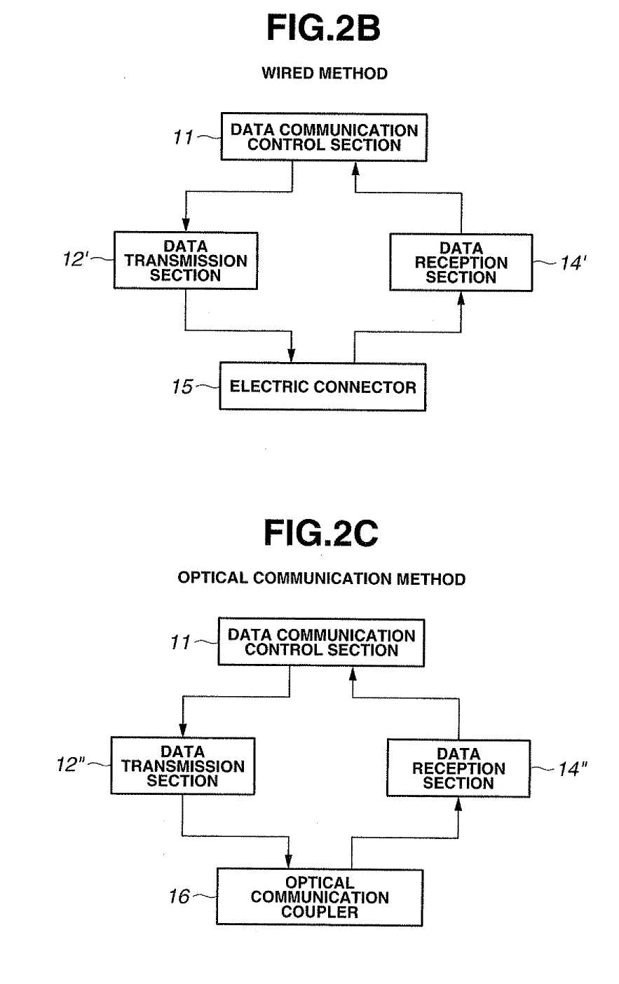

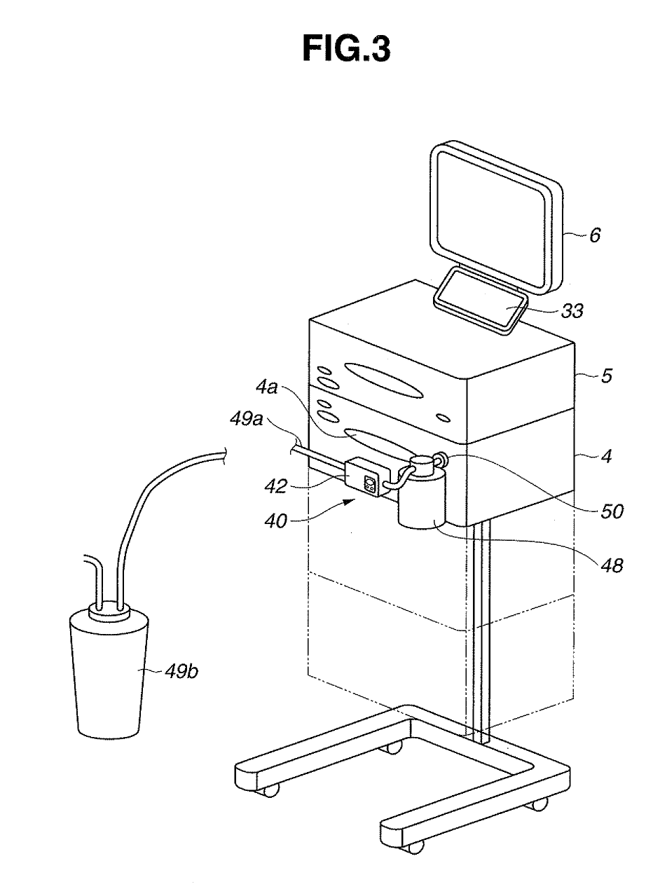

FIG. 1 shows a schematic structure of an endoscopic system according to the first embodiment of the present invention, FIGS. 2A to 2C are block diagrams showing each example of data communication mode in the endoscopic system according to the first embodiment, and FIG. 3 is a perspective view showing a specific outer appearance shape of an AWS unit peripheral section in the endoscopic system according to the first embodiment.

As shown in FIG. 1, an endoscopic system 1 includes a flexible endoscope (also referred to as scope) 3 for performing an endoscopic inspection by inserting the endoscope in a body cavity of a patient (not shown) lying on an inspection bed 2. The endoscopic system 1 includes an air water supply/suction unit having functions of airing, watering, and suction (hereinafter, abbreviated as AWS unit) 4, an endoscopic system control device 5 for performing a signal process on an image pickup element built in the endoscope 3 and a control process, an image pickup process, and the like on various operation sections provided to the endoscope 3, and an observation monitor 6 for displaying a video signal generated by the endoscopic system control device 5 such as a liquid crystal monitor, which are connected to the endoscope 3. It should be noted that the observation monitor 6 includes a touch panel 33.

In addition, the endoscopic system 1 further includes an image recording unit 7 for filing digital video signals which are, for example, generated by the endoscopic system control device 5, and a UPD coil unit 8 connected to the AWS unit 4, for displaying, when shape detecting coils (hereinafter, abbreviated as UPD coil) are built in the insert section of the endoscope 3, a shape of the insert section of the endoscope 3 by detecting each position of the UPD coils while a magnet field is received by the UPD coil.

In the case of FIG. 1, the UPD coil unit 8 is provided while embedded in an upper surface of the inspection bed 2. Then, the UPD coil unit 8 is connected to the AWS unit 4 via a cable 8a.

Also, in this embodiment, an accommodating concave portion is formed at one end section in the longitudinal direction of the inspection bed 2 and a position below the end section, whereby the tray conveyance trolley 38 can be accommodated. An endoscope tray 39 for accommodating the endoscope 3 is placed on an upper part of the tray conveyance trolley 38.

Then, the endoscope tray 39 accommodating the endoscope 3 after being subjected to sterilization or disinfection can be conveyed by the tray conveyance trolley 38 to be accommodated in the accommodating concave portion of the inspection bed 2. The surgeon can use the endoscope 3 by removing from the endoscope tray 39 and also can accommodate the endoscope 3 in the endoscope tray 39 after the end of the endoscopic inspection again. After that, with use of the tray conveyance trolley 38, sterilization or disinfection can be performed smoothly as well by conveying the endoscope tray 39 accommodating the used endoscope 3.

Then, the AWS unit 4 and the endoscopic system control device 5 shown in FIG. 1 wirelessly perform information transmission and reception in this embodiment. It should be noted that in FIG. 1, the endoscope 3 is connected to the AWS unit 4 via a tube unit 19, but wireless information transmission and reception (bidirectional transmission) may be performed. The endoscopic system control device 5 may wirelessly perform information transmission and reception with the endoscope 3.

FIGS. 2A to 2C show three methods in a transmission and reception unit (communication section) for performing data transmission and reception between a unit and a device in the endoscopic system 1 or between the endoscope 3 and a unit or a device. In FIG. 2A, as a specific example, the case of the AWS unit 4 and the endoscopic system control device 5 will be described.

FIG. 2A shows a wireless method, in which with a data communication control section 11 built in the AWS unit 4, transmission data is modulated via a data transmission section 12 and wirelessly sent to the endoscopic system control device 5 from an antenna section 13.

Then, the AWS unit 4 receives the wirelessly transmitted data from the endoscopic system control device 5 side at the antenna section 13, and sends the data which is demodulated by a data reception section 14, to the data communication control section 11. According to the present invention, when the data is transmitted in the wireless method, a wireless LAN is formed which has the maximum data communication speed of 54 Mbps on the basis of the IEEE802.11g standard, for example.

FIG. 2B shows a wired method. As a specific example, a case of performing data transmission and reception between the endoscope 3 and the AWS unit 4 will be described. With the data communication control section 11 built in the endoscope 3, the data transmitted from the endoscope 3 is received via a data transmission section 12′ by the AWS unit 4 from an electric connector 15 in a wired way. Then, the data transmitted from the AWS unit 4 is sent via the electric connector 15 and a data reception section 14′ to the data communication control section 11.

FIG. 2C shows an optical communication system. As a specific example, a case of performing data transmission and reception between the AWS unit 4 and the endoscopic system control device 5 will be described. The data communication control section 11 built in the AWS unit 4 is connected, via a data transmission section 12″ and a data reception section 14″ for performing transmission and reception with use of light, to an optical communication coupler 16 provided in the AWS unit 4. The data communication control section 11 then performs data transmission and reception via an optical communication coupler on the endoscopic system control device 5 side.

As shown in FIG. 1, the endoscope 3 of the first embodiment includes an endoscope main body 18 and the disposal tube unit 19 detachably connected to the endoscope main body 18.

The endoscope main body 18 includes an elongate flexible insert section 21 inserted in the body cavity, and an operation section 22 provided at a rear end of the insert section 21. A base end of the tube unit 19 is detachably connected to the operation section 22.

In addition, an image pickup unit is arranged at a distal end section 24 of the insert section 21 as the image pickup element. The image pickup unit uses a charged coupled device (abbreviated as CCD) 25 for varying a gain inside the image pickup element.

A bending section 27 which can be bent with a small power is provided at a rear end of the distal end section 24. By operating a track ball 69 as an operation section (instruction input section) provided at the operation section 22, the bending section 27 can be bent. The track ball 69 is also used for the articulation operation (bending operation) and changing and setting of other endoscope switch functions, for example, setting of articulation sensitivity, airing amount, and the like.

Consistency varying sections including consistency varying actuators 54A and 54B which are consistency variable are formed at plural locations of the insert section 21, whereby an insert operation or the like can be smoothly performed.

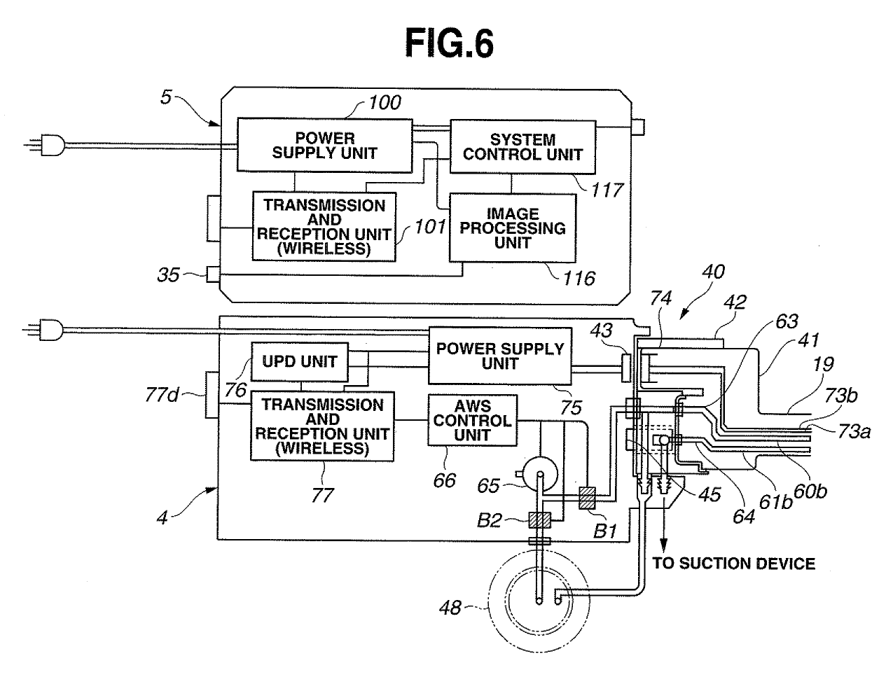

In this embodiment, the AWS unit 4 and the endoscopic system control device 5 side perform the data transmission and reception, for example, with wireless transmission and reception units 77 and 101, as shown in FIG. 6. Then, the observation monitor 6 is connected to a monitor connector 35 of the endoscopic system control device 5 with use of a monitor cable.

As will be described later, image data captured by a CCD 25 from the AWS unit 4 side and image data of the insert section shape of the endoscope 3 detected by using the UPD coil unit 8 (the UPD image) are transmitted to the endoscopic system control device 5. Thus, the endoscopic system control device 5 transmits video signals of the image data to the observation monitor 6, whereby the UPD image can be also displayed with the endoscope image on the display screen.

The observation monitor 6 is composed of a monitor of a high definition TV (HDTV) so that images of plural types can be displayed on the image screen at the same time in this way.

Furthermore, as shown in FIG. 1, for example, the AWS unit 4 includes an endoscope connector 40. Then, an endoscope connector 41 of the endoscope 3 is detachably connected to the endoscope connector 40.

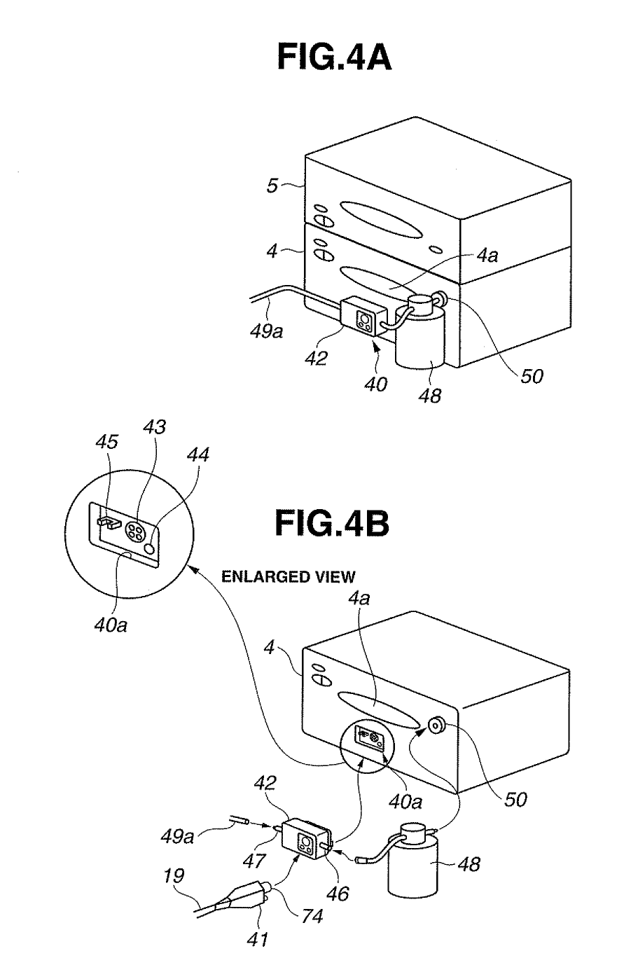

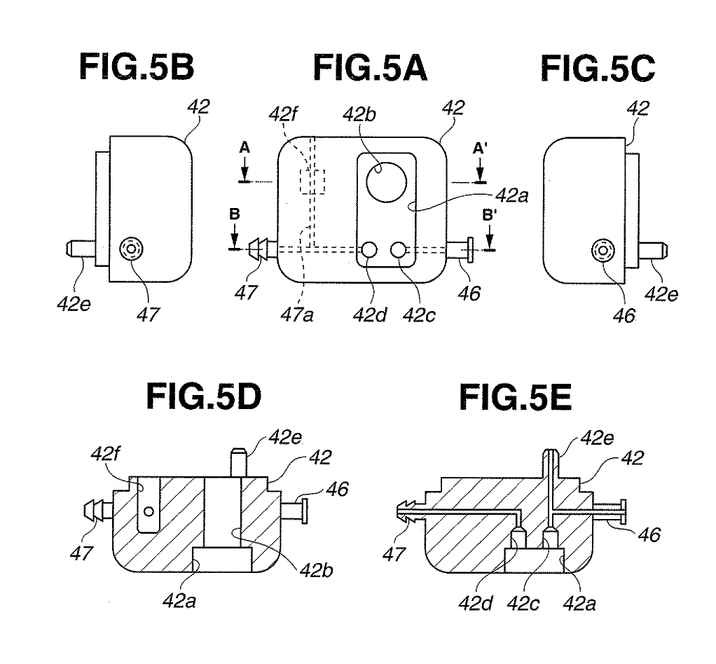

In this case, FIGS. 3 and 4 show an outer appearance shape of the endoscope connector 40 on the AWS unit 4 side. Then, FIGS. 5A to 5E show a structure of an AWS adapter 42 detachably attached to the endoscope connector 40 of the AWS unit 4, and FIG. 6 shows internal structures of the endoscope connector 40 on the AWS unit 4 side and the endoscope connector 41 on the endoscope 3 side by way of connection status.

In actuality, as shown in FIG. 4B, a concave AWS adapter attachment section 40a is provided on the front face of the AWS unit 4. An AWS adapter (duct line connection adapter) 42 shown in FIGS. 5A to 5E is attached to the AWS adapter attachment section 40a, thereby forming the endoscope connector 40, and the endoscope connector 41 on the endoscope 3 side is connected to the endoscope connector 40.

The AWS adapter attachment section 40a includes an endoscope electrical connector 43, an airing connector 44, and a pinch valve 45. An inner end surface of the AWS adapter 42 is detachably attached to the AWS adapter attachment section 40a, and from an outer end surface side, the endoscope connector 41 of the endoscope 3 is connected.

A detail of the AWS adapter 42 is shown in FIGS. 5A to 5E. FIG. 5A is a front view of the AWS adapter 42, FIGS. 5B and 5C are left and right side views, FIGS. 5D and 5E are cross-sectional views taken along the lines A-A′ and B-B′ in FIG. 5A, respectively.

The endoscope connector 41 is inserted to a concave portion 42a on the front face of the AWS adapter 42. In that case, an electrical connector section of the endoscope connector 41 is inserted to a through hole 42b provided in the concave portion and connected to the facing endoscope connector 43 in the through hole 42b.

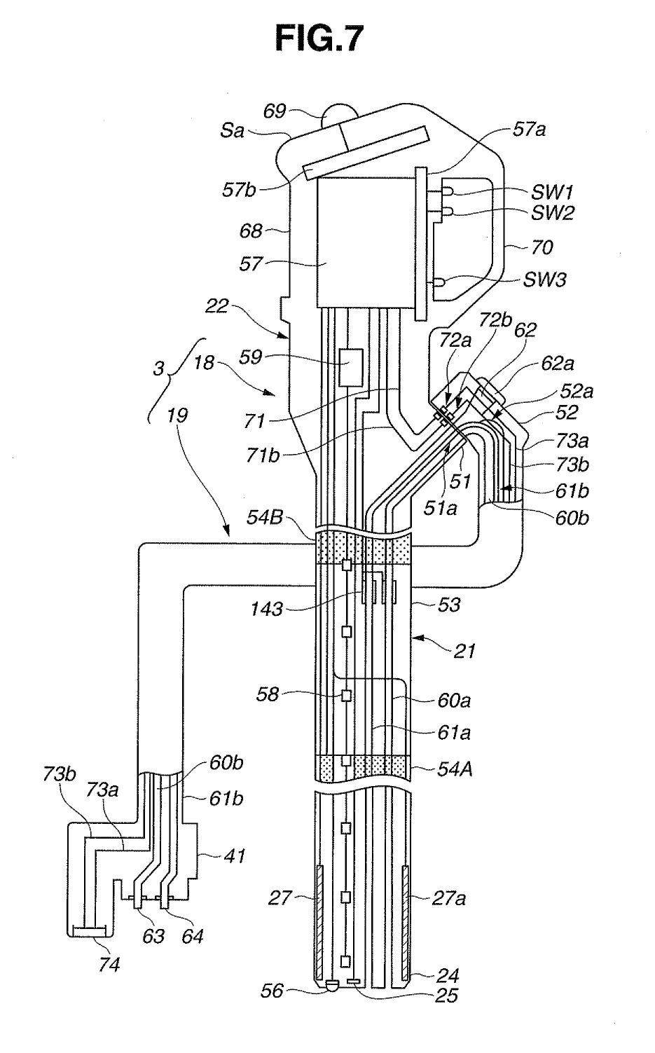

An air water connecter 42c and a suction connecter 42d are provided on a lower side of the through hole 42b, to which an air water connecter 63 and a suction connecter 64 in the endoscope connector 41 (refer to FIGS. 6 and 7) are connected, respectively. It should be noted that a concave portion 42f is provided on a base end surface side of the AWS adapter 42 for accommodating the pinch valve 45 protruding from the AWS adapter attachment section 40a.

As shown in FIG. 5E, the air water connecter 42c provided to the AWS adapter 42 has an internal duct line in communication therewith is branched, thereby forming an air connecter 42e connected to the airing connector 44 of the AWS unit 4 and a water connecter 46. Also, in the suction connecter 42d, a duct line in communication therewith is bent to protrude from the side to form a suction connecter 47 and a relief duct line 47a is also formed by being branched towards the upper side in the middle way, for example. After the relief duct line 47a is pinched by the pinch valve 45 in the middle way, the upper end is opened.

When a suction pump not shown forming the suction section is set in a regular operation status, the relief duct line 47a is normally set in a released status by the pinch valve 45, and when a suction operation is performed, the pinch valve 45 is driven. Then, as the relief duct line 47a is closed by the pinch valve 45, the release is cancelled, and the suction operation can be performed.

The water connecter 46 and the suction connecter 47 are connected, as shown in FIG. 3 or the like, to a watering tank 48 and a suction device (inserted by a suction tank 49b via a suction tube 49a), respectively. The watering tank 48 is connected to a watering tank connecter 50 of the AWS unit 4. It should be noted that an operation panel 4a is provided on the upper side of the endoscope connector 40 in the front face of the AWS unit 4.

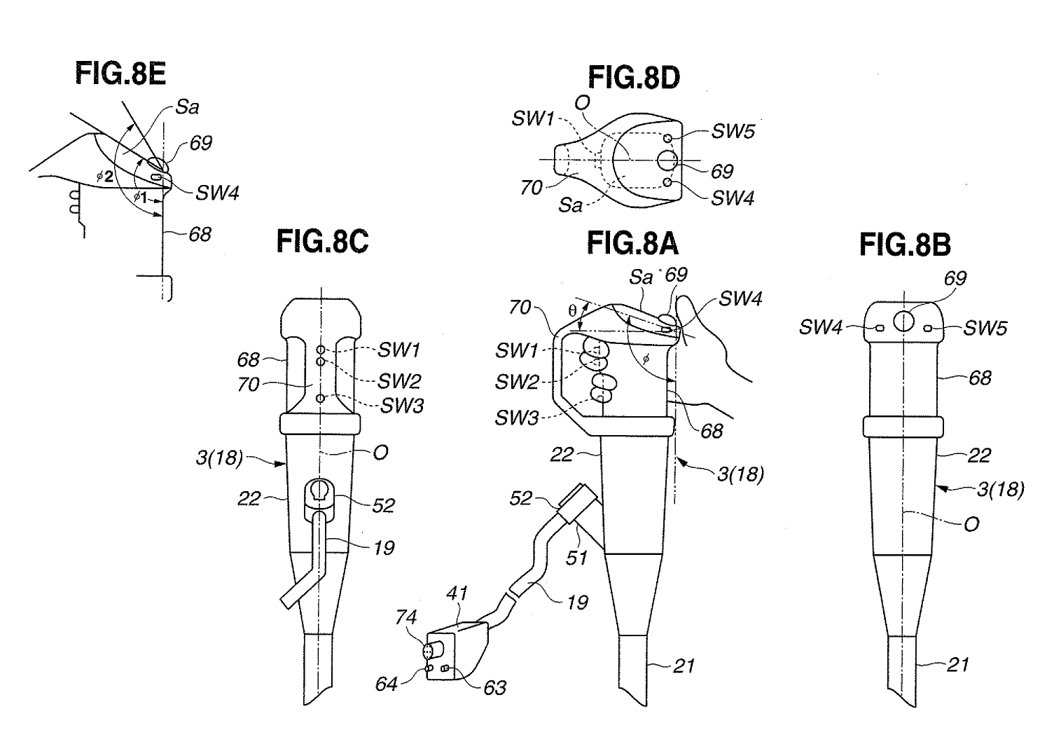

Next, with reference to FIGS. 7 and 8, a specific structure of the endoscope 3 of the first embodiment of the present invention will be described.

It should be noted that FIG. 8A shows a vicinity of the operation section of the endoscope 3 from a side, FIG. 8B is a front view as seen from the right hand side of FIG. 8A, FIG. 8C is a back view as seen from the left hand side of FIG. 8A, and FIG. 8D is a plan view as seen from the top of FIG. 8A. Then, FIG. 8E shows an example of an optimal angle range of the inclined surface.

In FIG. 1, as the outline has been described, the flexible endoscope 3 includes the endoscope main body 18 having the elongated flexible insert section 21 and the operation section 22 provided at the rear end, and the disposal tube unit 19 having a connecter section 51 (for the tube unit connection) in the vicinity of a base end (front end) of the operation section 22 in the endoscope main body 18, to which an overall connecter section 52 at the base end is detachably connected.

The endoscope connector 41 detachably connected to the AWS unit 4 described above is provided to a tail end of the tube unit 19.

The insert section 21 includes the rigid distal end section 24 provided at the distal end of the insert section 21, the freely bendable bending section 27 provided at the rear end of the distal end section 24, and an elongated flexible portion (corrugated tube section) 53 extending from the rear end of the bending section 27 to the operation section 22. The consistency varying actuators 54A and 54B formed of an electroconductive polymer artificial muscle (abbreviated as EPAM) or the like which expands upon voltage application while the consistency can be changed) are provided at plural positions, to be specific, two locations, in the middle way of the flexible portion 53.

For example, a light emitting diode (abbreviated as LED) 56 is arranged as the illumination section on an inner side of an illumination window provided to the distal end section 24 of the insert section 21. The illumination light of the LED 56 is output forward via an illumination lens integrally attached to the LED 56 for illuminating a subject such as an affected area. It should be noted that the light emitting element forming the illumination section is not limited to the LED 56, and the illumination section can be formed by using an LD (laser diode) or the like.

An objective lens not shown is attached to the observation window adjacently provided to this illumination window, and the CCD 25 including the gain varying function is arranged at the image forming location, thereby forming the image pickup section for capturing the subject.

The signal line that is inserted in the insert section 21 and whose ends are connected to the LED 56 and the CCD 25, respectively, is provided in the operation section 22 and connected to the control circuit 57 for performing a central control process (collective control process).

The UPD coils 58 are arranged at plural positions along the longitudinal direction in the insert section 21, and the signal line connected to each of the UPD coils 58 is connected to the control circuit 57 via a UPD coil driver unit 59 provided in the operation section 22.

Also, an articulation actuators 27a formed by arranging EPAM in the longitudinal direction as articulation elements (bending elements) are arranged at four locations in the circumferential direction on the inner side of an outer skin in the bending section 27. The articulation actuator 27a and the consistency varying actuators 54A and 54B are also connected to the control circuit 57 via the signal line. The control circuit 57 is structured by mounting, for example, electronic circuit elements on a switch board 57a and a track ball board 57b.

The EPAM used for the articulation actuator 27a and the consistency varying actuators 54A and 54B has, for example, electrodes attached on board-shaped both sides. With the application of a voltage, contraction in a thickness direction is caused, whereby expansion in the longitudinal direction can be achieved. It should be noted that this EPAM can vary a warpage, for example, in proportion to a about square of the applied voltage.

When used as the articulation actuator 27a, the EPAM is formed into a wire to expand on one side and contract on the other side, thereby bending the bending section 27 similarly to the normal wire function. Also, with the expansion or contraction, the consistency can be varied. By using the functions of the consistency varying actuators 54A and 54B, the consistency of that part can be varied.

An air water duct line 60a and a suction duct line 61a are inserted through the insert section 21, and the rear end functions as a duct line connector 51a which is opened in the connecter section 51. Then, a duct line connector 52a in the overall connecter section 52 at the base end of the tube unit 19 is detachably connected to the duct line connector 51.

Then, the air water duct line 60a is connected to an air water duct line 60b inserted through the tube unit 19. The suction duct line 61a is connected to a suction duct line 61b inserted through the tube unit 19 and is branched in the duct line connector 52a to have an opening at the outside, which is in communication with an insertion port (also referred to as biopsy port) 62 for allowing insertion of an endo-therapy product such as forceps. The biopsy port 62 is closed by a forceps valve 62a when not used. The rear ends of the air water duct line 60b and the suction duct line 61b function as the air water connecter 63 and the suction connecter 64 in the endoscope connector 41.

The air water connecter 63 and the suction connecter 64 are connected to the air water connecter 42c and the suction connecter 42d of the AWS adapter 42 shown in FIGS. 4 and 5 and the like, respectively. Then, as shown in FIGS. 5A to 5E, the air water connecter 42c is branched into the air duct and the water duct line inside the AWS adapter 42. The air duct is connected to an airing pump 65 in the AWS unit 4 via an electromagnetic valve B1, whereas the water duct line is connected to the watering tank 48. Also, the watering tank 48 is connected to the airing pump 65 via an electromagnetic valve B2 in the middle way.

The airing pump 65 and the electromagnetic valves B1 and B2 are connected to the AWS control unit 66 through a control line (driver line). With the AWS control unit 66, closing and opening are controlled, whereby airing and watering can be conducted. It should be noted that the AWS control unit 66 performs an operation control for suction under the control of opening and closing of the pinch valve 45.

Then, the operation section 22 of the endoscope main body 18 includes a grasping section 68 grasped by the surgeon. In this embodiment, as shown in FIGS. 8A to 8D, the grasping section 68 is formed of a side part of a cylindrical shape, for example, in the vicinity of the rear end (base end) in the operation section 22 (on the opposite side to the insert section 21).

The peripheral section including the grasping section 68 has, for example, three endoscope switches SW1, SW2, and SW3 for performing remote control operations such as release and freeze (abbreviated as remote control operations) arranged in the axis of the longitudinal direction of the operation section 22, which are connected to the control circuit 57 (refer to FIG. 7).

Furthermore, a base end surface at the rear end (base end) of the grasping section 68 (or the operation section 22) (referred to as upper end surface in general, because the base end side is set as the up direction as shown in FIGS. 8A to 8E to be used in the endoscopic inspection) is formed to be an inclined surface Sa. The track ball 69 of a water proof structure for performing the articulation operation (bending operation) and setting of other remote control operation by switching from the articulation operation is provided in the vicinity of the opposite side to the locations where the endoscope switches SW1, SW2, and SW3 are provided in the inclined surface Sa. It should be noted that the water proof structure in this case refers to, in actuality, a structure in which the encoder side for rotatably holding the track ball 69 and detecting the rotation amount of the track ball 69 is covered with a water proof coating, and the track ball 69 is rotatably held outside thereof.

In addition, a substantially U-shaped hook 70 is provided for connecting vicinities of both the end of the longitudinal direction in the grasping section 68 provided in the vicinity of the rear end of the operation section 22. As shown in FIG. 8B, the surgeon puts a finger of the hand into the inside of the hook 70 for grasping by right hand (or left hand), even in the case in which the grasping section 68 is not firmly grasped, the endoscope 3 can be effectively prevented from dropping due to the weight.

In other words, even if the endoscope 3 tries to drop due to the weight, a lower side of the hook 70 is touched by the hand, whereby the drop of the endoscope 3 can be prevented. In this way, in this embodiment, even when the surgeon does not grasp (hold) the grasping section 68 firmly, the endoscope 3 can be effectively prevented from dropping down due to the weight. Therefore, in the case where the surgeon performs various operations while grasping the grasping section 68 and the surgeon is tired from using the grasping hand or finger for the operations, even when the surgeon stops grasping (holding) the grasping section 68, if the surgeon puts a part of the hand into the hook 70, the drop of the endoscope 3 or the like can be prevented, so the operability can be improved.

Also, as shown in FIGS. 8A to 8C, the air water switch SW4 and the suction switch SW5 are bilaterally symmetrically arranged on both sides of the track ball 69 in the inclined surface Sa.

The track ball 69 and the endoscope switches SW4 and SW5 are also connected to the control circuit 57. As will be described further with reference to FIGS. 8A to 8D, the operation section 22 or the grasping section 68 has a shape bilaterally symmetric to a center line O (as the reference line) extending in the longitudinal direction of the operation section 22 or the grasping section 68 in the front view shown in FIG. 8B. The inclined surface Sa at a location on the center line O has the track ball 69 arranged. Then, the air water switch SW4 and the suction switch SW5 are arranged at bilaterally symmetric positions on both the sides of the track ball 69.

A back view on the opposite side to this front view is FIG. 8C. In this back view too, the three endoscope switches SW1, SW2, and SW3 are arranged on the outer surface of the grasping section 68 so as to be bilaterally symmetric with respect to the center line O on the center line O.

Also, in this embodiment, as shown in FIG. 8A, the inclined surface Sa is formed to have an angle . which is an angle larger than 90°, or an obtuse angle, with respect to a parallel line to the center line O or the side face of the grasping section 68. In other words, the inclined surface Sa is formed to be an inclined surface to have an angle θ with respect to a surface perpendicular to the center line O of the grasping section 68. The track ball 69, and the air water switch SW4, and the suction switch SW5 are bilaterally symmetrically provided at low section side positions in the inclined surface Sa. Then, as shown in FIG. 8B, with a thumb of the grasping hand, the track ball 69 or the like can be easily operated.

As described above, the inclined surface Sa can be operated when an angle . is an obtuse angle to the center line O, in other words, from 90° to 180°. More specifically, as shown in FIG. 8E, if the angle is from 120° that is an angle 0.1 to 150° that is an angle 0.2, a farther satisfactory operability can be ensured.

In this manner, in this embodiment, the operation section (instruction input section) such as the track ball 69 provided to the operation section 22 is arranged bilaterally symmetric to the center line O in the longitudinal direction of the grasping section 68, thereby attaining one feature related to the satisfactory operation even when the surgeon grasps by using the right hand or the left hand.

Also, the grasping section 68 includes the hook 70 for connecting about both the ends in the longitudinal direction of the grasping section 68 by forming the ends into a substantially U-shape. Even if the surgeon does not sufficiently grasp the grasping section 68, as the index finger or the like is inserted inside the hook 70, when the endoscope 3 drops down due to the weight, the hook 70 is hooked by the index finger or the like, thereby providing the function of effectively preventing the drop of the endoscope 3.

Also, in this embodiment, the grasping section 68 is formed in the vicinity of the rear end of the operation section 22, and a connection section with the tube unit 19 is provided at a position nearer to the insert section 21 than the grasping section 68. Thus, it is possible to reduce the effect of eccentricity of the gravity center of the grasping section 68 when grasped, from the position of the center axis.

In other words, if the tube unit 19 is extended to the side from the rear side (upper side) position than the grasping section position of the prior art, the position of the gravity center at that case is easy to decenter due to the weight of the tube unit. In this embodiment, the tube unit 19 is extended from a position nearer to the insert section 21 than the grasping section 68, in other words, toward the side from the position on the lower side. Thus, the eccentricity of the gravity center position can be reduced, and the operability can be improved.

Then, in the endoscope 3 of this embodiment as well, when the operator (the user) such as the surgeon grasps the grasping section 68 by the left hand or the right hand, such a state that the inner surface side of the hook 70 is lightly touched by an area in the vicinity of the side section of the index finger. Even if the gravity center position is decentered to cause an effect that the center axis is inclined, (in other words, the longitudinal direction of the operation section 22 is inclined), the hook 70 is touched by the hand and the inclination is restricted, whereby the satisfactory operability can be ensured.

As shown in FIG. 7, a power supply line 71a and a signal line 71b extending from the control circuit 57 are connected via electromagnetic coupling connection sections 72a and 72b to a power supply line 73a and a signal line 73b that are formed in the connecter section 51 and the overall connecter section 52 and inserted through the tube unit 19 by way of electromagnetic coupling. The power supply line 73a and the signal line 73b are connected to a power supply and signal terminal which form an electrical connector 74 in the endoscope connector 41.

Then, while the user connects the endoscope connector 41 to the AWS unit 4, as shown in FIG. 6, the power supply line 73a is connected to the power supply unit 75 via the endoscope electrical connector 43 of the AWS unit 4, and the signal line 73b is connected (via the power supply unit 75) the UPD unit 76, the transmission and reception unit 77, and the AWS control unit 66. It should be noted that the transmission and reception unit 77 is connected to the antenna section 77a for performing wireless transmission and reception of radio waves.

It should be noted that the electromagnetic coupling connection sections 72a and 72b form a transformer for causing one pair of coils to be adjacent to each other and electromagnetically coupled. That is, the end section of the power supply line 71a is connected to a coil forming the electromagnetic coupling connection section 72a, and the other end section of the power supply line 73a is also connected to a coil adjacent to the coil in the electromagnetic coupling connection section 72a.

Then, the alternating current power transmitted by the power supply line 73a is sent via a coil electromagnetically coupled in the electromagnetic coupling connection section 72a to the power supply line 71a side.

The end section of the signal line 71b is connected to a coil forming the electromagnetic coupling connection section 72b, and the end of the other signal line 73b is also connected to a coil adjacent to the coil in the electromagnetic coupling connection section 72b.

Through the electromagnetic coupling for forming the transformer, the signal is transmitted via the pair coils from the signal line 71b side to the signal line 73b side, and the signal transmission in the opposite direction is also performed.

In this manner, the endoscope 3 of this embodiment has the structure in which the endoscope main body 18 is detachably connected to the tube unit 19, and even when washing, sterilization, and the like are repeatedly performed, there is attained a feature in that the effect of corrosion or the like generated at the time of mutual connection between metal electrodes can be prevented.

In addition, as shown in FIG. 7, transparency sensors 143 are provided in the midway of the air water duct line 60a and the suction duct line 61a, making it possible that each duct line of the air water duct line 60a and the suction duct line 61a formed of a transparent tube is transmitted with light to detect the contamination degree of the inner wall of the duct line and the transparency of fluid passing through the inside of the duct line.

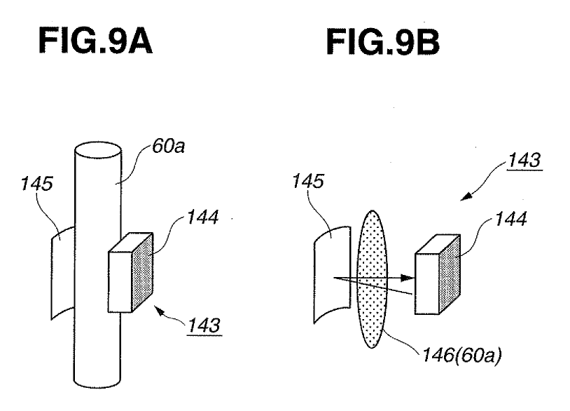

The transparency sensor 143 is connected to the control circuit 57 with a signal line. FIGS. 9A and 9B are explanatory diagrams for the effect of the washing level detection by the transparency sensor 143.

As shown in FIG. 9A, a photo reflector 144 and the reflection mirror 145 are arranged so as to oppose to each other on the outer periphery of the air water duct line 60a (as in the suction duct line 61a) formed of a transparent tube, thereby forming the transparency sensor 143.

Then, as shown in FIG. 9B, the light emitted by the light emitting element forming the photo reflector 144 is output to the reflection mirror 145 side, and the reflection light reflected by the reflection mirror 145 is received by a light reception element forming the photo reflector 144.

In this case, in actuality, as a transmittance detection body 146 such as the air water duct line 60a formed of a transparent tube is arranged between the photo reflector 144 and the reflection mirror 145, when a transparent washing liquid is poured into the inner wall side of the air water duct line 60a to wash the air water duct line 60a, once the inner wall surface is in a clean state, the light reception element of the photo reflector 144 receives larger light quantity, so the washing degree can be detected. Therefore, with this function, the washing level of the inner wall surface of the air water duct line 60a and that of the inner wall surface of the suction duct line 61a can be quantitative detected.

It should be noted that with the description in this case, the effect in the washing with the washing liquid is described, but during the endoscopic inspection or the like, by referring to the detection output from the transparency sensor 143, it is also possible to find out the contamination degree of the inner wall of the air water duct line 60a and that of the inner wall of the suction duct line 61a.

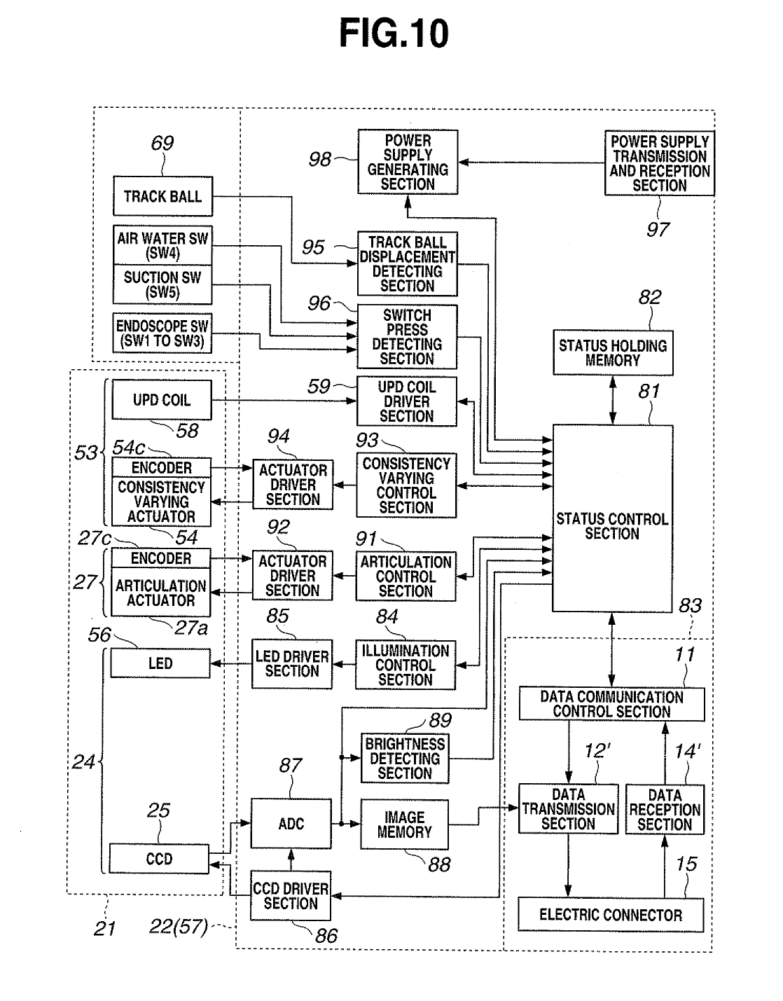

FIG. 10 shows the control circuit 57 and the like arranged the operation section 22 of the endoscope main body 18 and a structure of an electrical system of main component elements arranged at the respective sections of the insert section 21.

The CCD 25 and the LED 56 are arranged at the distal end section 24 of the insert section 21 shown in the lower section on the left hand side of FIG. 10. The articulation actuator (in this embodiment, specifically, EPAM) 27a and an encoder 27c are arranged at the bending section 27 shown in the upper section in the drawing. The consistency varying actuator (In this embodiment, specifically, EPAM) 54 and an encoder 54c are arranged at the flexible portion 53 shown in the upper section in the drawing. In addition, the transparency sensor 143 and the UPD coil 58 are arranged in the flexible portion 53.

Moreover, the track ball 69, the air water SW (SW4), the suction SW (SW5), and the endoscope SW (SW1 to SW3) are arranged on the surface of the operation section 22 in the upper section of the flexible portion 53 in the insert section 21. It should be noted that as will be described later, with the operation of the track ball 69, functions of selecting and setting the articulation operation and other functions are allocated.

As shown in the left hand side of FIG. 10, these are connected to via the signal line to the control circuit 57 including most of the inside of the operation section 22 shown on the right hand side (but except for the UPD coil driver unit 59 and the like), and the control circuit 57 performs the drive control for the functions, a signal processing, etc.

The control circuit 57 includes the status control section 81 composed of the CPU for managing the control status and the like. The status control section 81 is connected to the status holding memory 82 for holding (storing) the status of the respective sections and also to the transmission and reception unit 83 of the wired method for performing wired communication with the AWS unit 4 (in this embodiment).

Then, the status control section 81 controls via an illumination control section 84 for controlling the illumination an LED driver section 85 that is controlled by the illumination control section 84. The LED driver section 85 applies the LED 56 with the LED driver signal to cause the LED 56 functioning as the illumination section to emit light.

With the light emittance of the LED 56, the illuminated subject such as the affected area is imaged on an image pickup surface of the CCD 25 located at the image location by an objective lens not shown attached to the observation window, and photoelectric conversion is performed by the CCD 25.

In response to the CCD driver signal application from a CCD driver section 86 controlled by the status control section 81, the CCD 25 outputs the signal charge accumulated through the photoelectric conversion in the form of the image pickup signal. The image pickup signal is converted from an analog signal to a digital signal by an A/D converter (abbreviated as ADC) 87 and then input to the status control section 81. At the same time, the digital signal (image data) is stored in an image memory 88. The image data in the image memory 88 is sent to the data transmission section 12′ of the transmission and reception unit 83.

Then, the image data is transmitted to the AWS unit 4 side from the electric connector 15 via the signal line 73b in the tube unit 19. Furthermore, the image data is wirelessly from the AWS unit 4 to the endoscopic system control device 5.

As shown in FIG. 6, the image data transmitted to the endoscopic system control device 5 is wirelessly received by the transmission and reception unit 101. A video signal is generated through the image processing by the image processing unit 116. The video signal is output from the monitor connector 35 to the observation monitor 6 via the system control unit 117 that controls the overall endoscopic system 1, whereby the endoscope image is displayed on the display screen of the observation monitor 6. It should be noted that in FIG. 6, a power supply unit 100 supplies the transmission and reception unit 101, the image processing unit 116, and the system control unit 117 with the operation power.

As shown in FIG. 10, an output signal of the ADC 87 is sent to a brightness detecting section 89. Information on the image brightness detected by the brightness detecting section 89 is sent to the status control section 81. The status control section 81 performs light intensity adjustment on the basis of this information, so that the illumination quantity by the LED 56 is set to an appropriate brightness via the illumination control section 84.

Then, the status control section 81 controls an actuator driver section 92 via the articulation control section 91 to perform a control for driving the articulation actuator (EPAM) 27a with the actuator driver section 92. It should be noted that the drive amount of the articulation actuator (EPAM) 27a is detected by the encoder 27c so that the drive amount is controlled to match the instructed amount.

The status control section 81 controls the actuator driver section 94 through the consistency varying control section 93. With the actuator driver section 94, the consistency varying actuator (EPAM) 54 (this reference numeral represents 54A and 54B herein) is controlled for the drive. It should be noted that the drive amount of the consistency varying actuator (EPAM) 54 is detected by the encoder 54c so that the drive amount is controlled to be a value corresponding to the drive amount.

A detection signal from the transparency sensor 143 provided in the flexible portion 53 is converted into signal data corresponding to the transparency by a transparency detection section 148, and is then input to the status control section 81. The status control section 81 compares the signal data with a reference value of the transparency previously stored in the status holding memory 82 or the like. When the signal data reaches the reference value, the information is transmitted from the transmission and reception unit 83 via the AWS unit 4 to the endoscopic system control device 5 side, and the observation monitor 6 displays that the signal data reaches the reference value.

Data corresponding to the operation amount from the track ball 69 or the like provided to the operation section 22 is input to the status control section 81 via a track ball displacement detecting section 95. Furthermore, the switch press operation such as turning ON of the air water SW, the suction SW, and the endoscope SW is detected by a switch press detecting section 96, the detected information is input to the status control section 81.

The control circuit 57 includes a power supply transmission and reception section 97 and a power supply generating section 98. The power supply transmission and reception section 97 specifically means a transmission unit 51b in the operation section 22 or the electrical connector 74 at the tail end of the tube unit 19. Then, the electric power transmitted from the power supply generating section 98 is converted into a direct current power supply in the power supply generating section 98. The power supply generated by the power supply generating section 98 supplies the respective sections with necessary electric power the control circuit 57 for the operation.

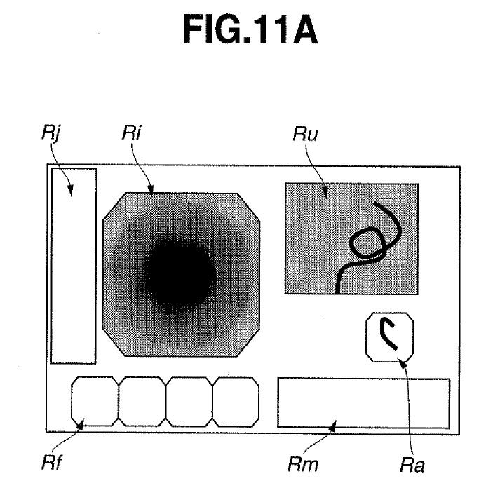

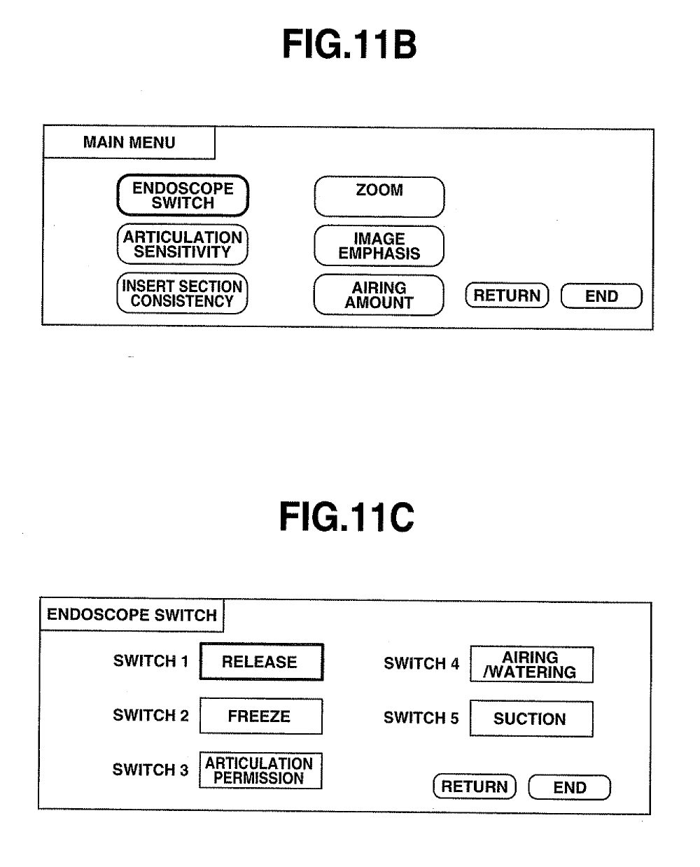

In the endoscopic system 1 of the first embodiment, when the power supply is activated, the observation monitor 6 displays various images shown in FIG. 11A, for example. In this case, in addition to an information display area Rj for displaying patient information or the like, a display area Ri of the endoscope image, a display area Ru of the UPD image, a display area Rf of a freeze image, and a display area Ra of an articulation shape, a menu display Rm is provided. The menu display Rm displays a menu.

As a menu displayed on the menu display Rm, a main menu shown in FIG. 11B is displayed. This main menu displays a return item for return operation instruction for returning to the previous menu screen and an end item for end, in addition to items of an endoscope switch, an articulation sensitivity, an insert section consistency, a zoom, an image emphasis, and an airing amount.

Then, when the user selects the endoscope switch item with a selection frame through the operation of the track ball 69 or the like, the frame of the endoscope switch item is displayed in bold and the display indicates the selected state. Furthermore, when the track ball 69 is pressed to perform a determined operation, whereby the functions to be allocated to the five switches SW1 to SW5 can be selected and set as shown in FIG. 11C.

Next, operation of the endoscopic system 1 of such a structure will be described.

As a prearrangement for the endoscopic inspection, first of all, the overall connecter section 52 of the disposal tube unit 19 is connected to the connecter section 51 in the operation section 22 of the endoscope main body 18. In this case, the electromagnetic coupling connection sections 72a and 72b are connected to each other in an insulated and water tight way. With this connection, the preparation of the endoscope 3 is completed.

Next, the endoscope connector 41 of the tube unit 19 is connected to a connector 40 of the AWS unit 4. With one touch connection of this part, various duct lines, the power supply line, the signal line, and optical connection are completed at once. Unlike the prior art endoscopic system, it is unnecessary to perform connection of various duct lines, connection of the electrical connector, and the like on each occasion.

In addition, the user connects the UPD coil unit 8 to the AWS unit 4, and connects the endoscopic system control device 5 to the observation monitor 6. If necessary, the endoscopic system control device 5 is connected to the image recording unit 7 or the like, thereby completing the setup of the endoscopic system 1.

Next, the power supplies of the AWS unit 4 and the endoscopic system control device 5 are turned ON. As a result, the respective sections are activated in the AWS unit 4, the power supply unit 75 can be in a status for supplying the endoscope 3 side with an electric power via the power supply line.

In this case, the AWS unit 4 firstly turns OFF the electric power supply, activates the timer, and confirms that a signal is returned from the endoscope 3 in a given period of time, and then continuously supplies the electric power.

Then, as the surgeon inserts the insert section 21 of the endoscope 3 in the body cavity of the patient, the subject such as the affected area in the body cavity is captured by the CCD 25 provided to the distal end section 24 of the insert section 21. The captured image data is wirelessly transmitted via the AWS unit 4 to the endoscopic system control device 5 to generate a video signal though image processing, whereby the subject image is displayed as the endoscope image on the display screen of the observation monitor 6. Therefore, while the surgeon observes the endoscope image, a diagnosis on the affected area or the like is performed, and treatment for a therapy can be also performed by using the endo-therapy product if necessary.

In the endoscope 3 of this embodiment, the track ball 69 having the function of the articulation instruction input section, the endoscope switches SW1 to SW3 for performing various operation instruction such as the freeze instruction operation, the air water switch (SW4), and the suction switch (SW5) are arranged bilaterally symmetrically to the center line O in the longitudinal direction of the grasping section 68 as shown in FIGS. 8A to 8E.

Therefore, as shown in FIG. 8B, for example, when the surgeon uses the right hand to grasp the grasping section 68 of the operation section 22, the track ball 69 is located at a position easy to be manipulated by the thumb, and the air water switch (SW4) and the suction (SW5) bilaterally symmetrically arranged can be easily operated.

Also, in the case of the grasping, the endoscope switches SW1 and SW2 are located in the vicinities of the grasping positions for the index finger and the middle finger, and further the endoscope switch SW3 is located in the vicinities of the grasping position for the little finger. Therefore, the surgeon can perform various operations with the satisfactory operability by the grasping right hand.

Then, in the case where the surgeon uses the left hand to grasp, the grasping position for grasping the grasping section 68 on the outer peripheral surface is on a side section side opposite to the side section grasped by the right hand. The positions for the respective fingers with respect to the instruction input sections are the same as those in the case of using the left hand. That is, when the surgeon uses the left hand to grasp the grasping section 68 of the operation section 22, the track ball 69 is located at the position easy to be operated by the thumb, and the air water switch (SW4) and the suction switch (SW5) which are bilaterally symmetrically arranged can be also operated.

In the case of grasping, the endoscopes SW1 and SW2 are arranged in the vicinities of the positions to be grasped by the index finger and the middle finger, and further the endoscope SW3 is arranged in the vicinity of the position to be grasped by the little finger. Therefore, the surgeon can perform can perform various operations by the grasping the left hand with the satisfactory operability.

As described above, in this embodiment, the hook 70 that links both the ends in the longitudinal direction of the grasping section 68 for allowing the grasping hand to pass through the inside is provided. Thus, even when the grasping section 68 is not firmly held, the endoscope 3 can be effectively prevented from dropping due to the weight.

Also, in this embodiment, as shown in FIGS. 11A to 11C, change setting of the function allocation for the endoscope switches SW1 to SW5 can be conducted. Therefore, each surgeon can also perform the endoscopic inspection in the most facilitated manner of the operations by changing and setting the function allocation for the endoscope switches SW1 to SW5.

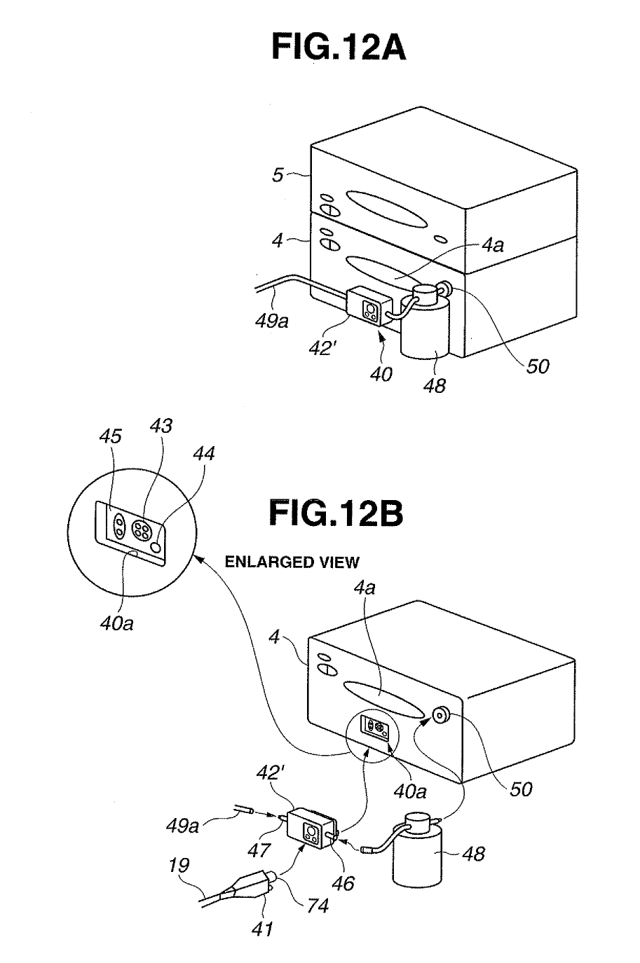

It should be noted that in the first embodiment, the description has been given of the structure where the pinch valve 45 is provided on the AWS unit 4 side, to which the AWS adapter 42 is connected, but as shown in FIGS. 12A and 12B, an electromagnetic valve unit 42′ may be detachably attached to the concave portion 40a of the AWS unit 4 (which is also a modified example of the AWS adaptor). Then, in the state where the electromagnetic valve unit 42′ is attached to the AWS unit 4, the endoscope connector 41 of the endoscope 3 is detachably mounted.

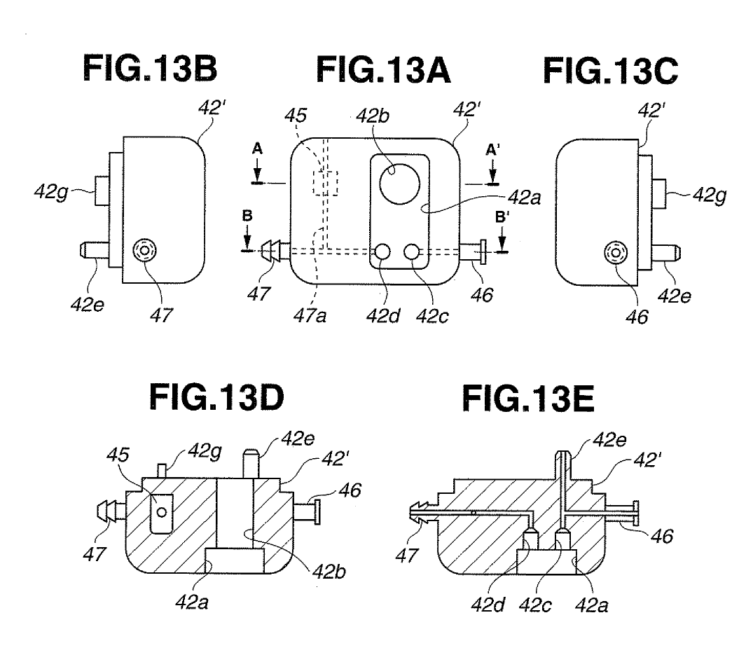

It should be noted that FIG. 13A is a front view of the electromagnetic valve unit 42′, FIGS. 13B and 13C are left and right side views, and FIGS. 13D and 13E are cross-sectional views taken by the lines A-A′ and B-B′ of FIG. 13A, respectively.

In the AWS adapter 42 of FIG. 5A to 5E, the concave portion 42f accommodating the pinch valve 45 (protruding from the front face of the AWS unit 4) is provided on the back (base end) side, but the electromagnetic valve unit 42′ shown in FIGS. 13A to 13E has the structure in which the pinch valve 45 is provided inside thereof, and the relief duct line 47a penetrates in the pinch valve 45.

Also, a pinch valve connector 42g that is detachably connected to the electromagnetic valve unit 42′ of the AWS unit 4 on the back side and transmits a signal for driving the pinch valve 45 is attached to the electromagnetic valve unit 42′. The other structure is the same as those of FIGS. 5A to 5E.

The operation effect in the case of adopting the AWS unit 4 and the electromagnetic valve unit 42′ in shown FIGS. 12 and 13 are almost the same as those of FIGS. 4 and 5.

Next, the endoscopic system according to a second embodiment of the present invention will be described.

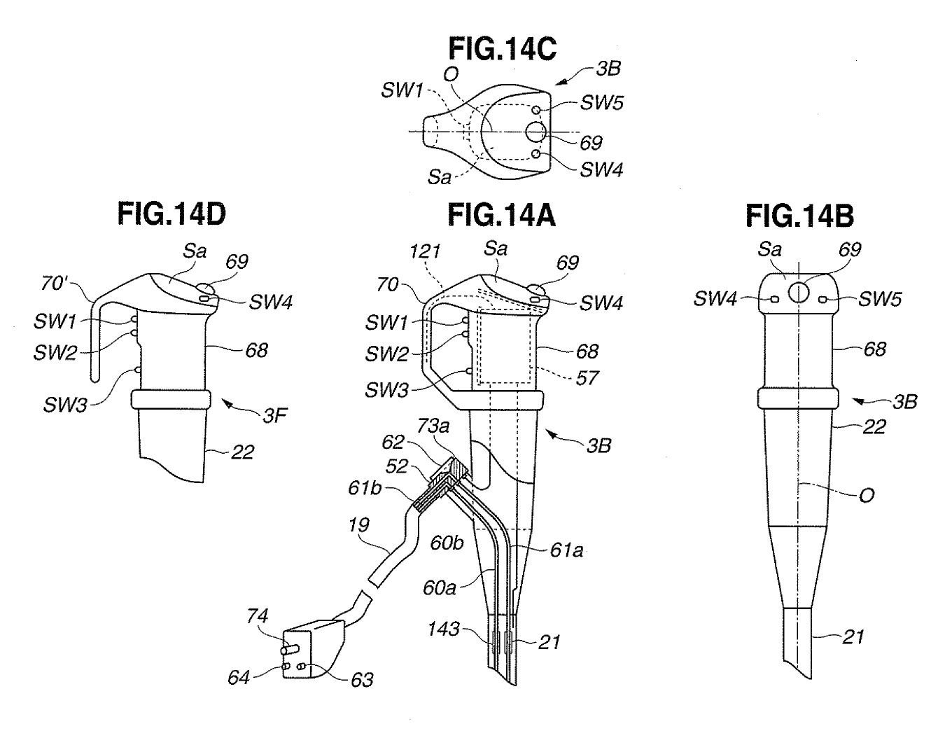

FIGS. 14A to 14E show a specific outer appearance shape or the like of the endoscope in the endoscopic system according to the second embodiment of the present invention.

It should be noted that FIG. 14A shows a state partially cut off from the side in the vicinity of the operation section, FIG. 14B is a front view as seen from the right hand side of FIG. 14A, FIG. 14C is a plan view as seen from the top of FIG. 14A, and FIG. 14D shows a part of the endoscope 3F as a modified example.

In the endoscope 3B according to the second embodiment, in the endoscope 3 of the first embodiment, an transmission and reception antenna section 121 built in the operation section 22 is used instead of providing the signal transmission signal line 73b.

Information such as image data captured by the CCD 25 or operation data in the case of operating the track ball 69 or the like as the operation section is transmitted to the AWS unit 4 side via the antenna section 121. The other structure is the same as that of the first embodiment.

In the endoscope 3B according to the second embodiment, the air water duct line 60b, the suction duct line 61b, and the power supply line 73a are inserted through the tube unit 19.

Also, according to this embodiment, the provision of the signal line 73b inserted through the tube unit 19 is unnecessary, so the structure more suitable to the disposal use can be obtained. Other structure is the same as that of the first embodiment. Even when the grasping section 68 of the endoscope 3B is grasped by a left or right hand of a right-handed or left-handed user, the operation can be conducted with the satisfactory operability.

FIG. 14D shows a first modification of the endoscope 3F. In the endoscope 3B of FIGS. 14A to 14C, the hook 70 links both the upper and lower end (in the longitudinal direction) in the grasping section 68 grasped by the hand in a loop way. However, according to a structure of the endoscope 3F, a hook 70′ is formed into an L shape from the upper end side of the grasping section 68, and the lower end of the hook 70′ is not linked to the grasping section 68, whereby an opening is formed in the lower end of the hook 70′.

In the case of this modified example too, the operation section 22 or the grasping section 68 is set to be bilaterally symmetric with respect to the center line O in the longitudinal direction, and at the same time the instruction input sections are formed so as to be bilaterally symmetric. Thus, the same operability can be ensured as in the case of the first embodiment or the second embodiment.

Also, the function of preventing the drop of the endoscope 3B which may occur when the grasping is not sufficiently performed is realized by the upper end section in the hook 70′, whereby almost the same function as that of the hook 70 can be maintained. That is, in the endoscope 3B as well, the hook 70′ is formed to have a protruding section protruding from the rear end side of the grasping section 68 in a direction perpendicular to the axis direction of the grasping section 68, thereby preventing the drop of the endoscope 3B effectively. In addition, as the hook 70′ protrudes in the direction perpendicular to the axis direction of the grasping section 68, and is formed into the L shape by bending into the insert section 21 side, so the drop of the endoscope 3B can be further effectively prevented.

Also, the lower end side of the hook 70′ is opened, so it is possible to hold the endoscope 3F by hooking this part to an endoscope hanger or the like. In this way, this modified example has almost the same operation effect as in the second embodiment.

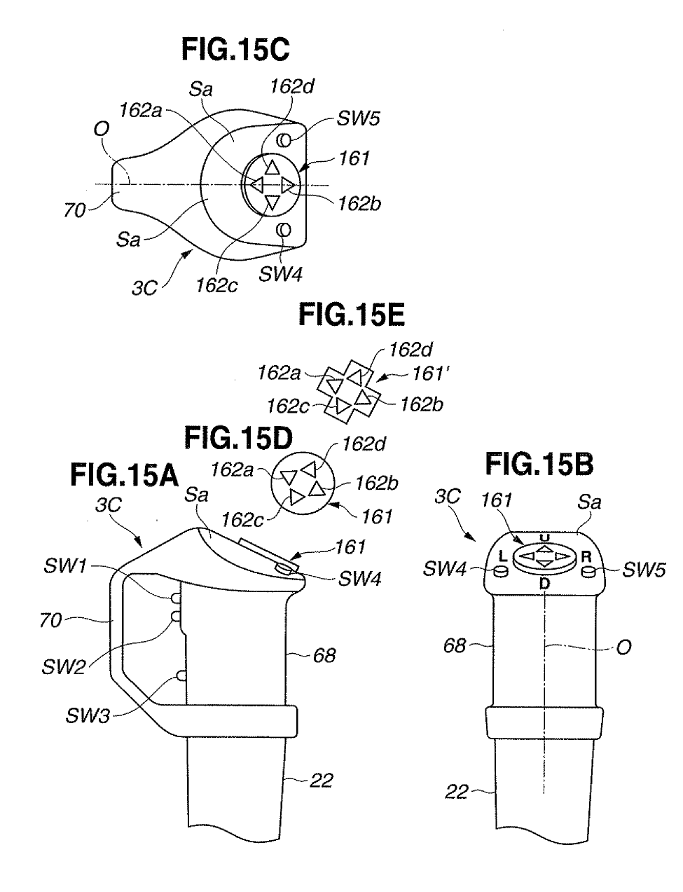

FIGS. 15A to 15E show an operation section and a peripheral section of a second modified example of the endoscope in the endoscopic system according to the second embodiment of the present invention.

FIGS. 15A to 15C show a second modified example of the endoscope 3. The endoscope 3C adopts the operation pad 161 instead of the track ball 69 as the operation section in the endoscope 3B according to the second embodiment.

It should be noted that FIG. 15A is a side view as seen from the side of the endoscope 3C, FIG. 15B is a front view as seen from the right hand side of FIG. 15A, FIG. 15C is a plan view as seen from the top of FIG. 15A, FIG. 15D shows the operation pad 161 as seen from a direction perpendicular to the inclined surface Sa in FIG. 15A in an arrangement state along the center line parallel to the inclined surface Sa, and FIG. 15E shown an operation pad 161′ of a modified example in the similar arrangement state as FIG. 15D.

The endoscope 3C adopts the disc-shaped operation pad 161 instead of the track ball 69 in the endoscope 3B shown in FIGS. 14A to 14D. That is, the operation pad 161 is attached to the inclined surface Sa. Switches 162a, 162b, 162c, and 162d for issuing operation instructions in four directions of up, down, left, and right are provided to the operation pad 161 at four positions corresponding to the four directions of up, down, left, and right.

The other structure is the same as that of the endoscope 3B shown in FIGS. 14A to 14D.

Alternatively, as a modified example of the second modified example of the operation pad 161A, a cross-shaped operation pad 161′ as shown in FIG. 15E may be adopted. The operation pad 161′ also has the switches 162a, 162b, 162c, and 162d for issuing operation instructions in the four directions of up, down, left, and right are provided at four positions corresponding to the four directions of up, down, left, and right.

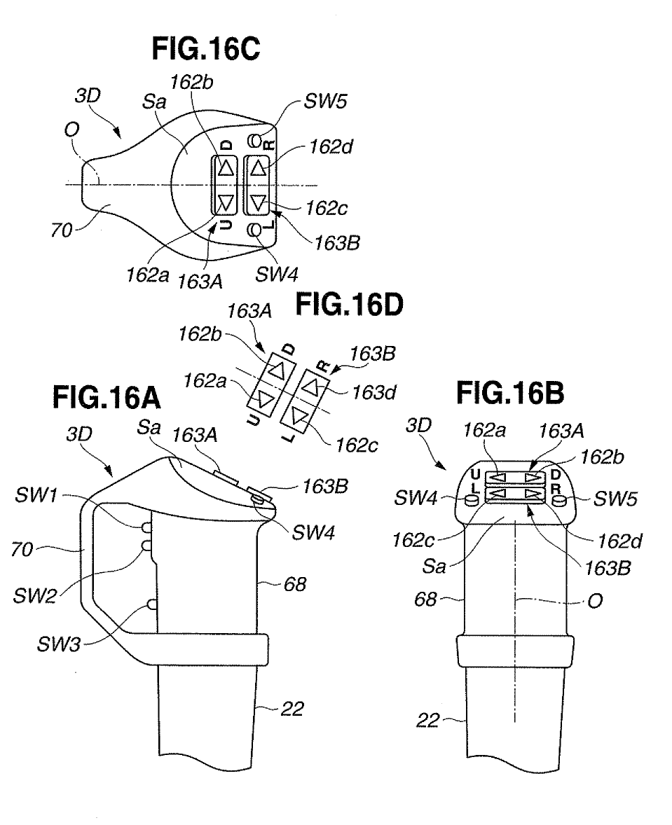

FIGS. 16A to 16D show an operation section and a peripheral section of a third modified example of the endoscope in the endoscopic system according to the second embodiment of the present invention.

FIGS. 16A to 16D show an endoscope 3D as the third modified example. For example, as shown in FIG. 16C, the endoscope 3D has two operation pads 163A and 163B in parallel at the position of the track ball 69 in the inclined surface Sa of the endoscope 3B shown in FIGS. 14A to 14D, in a direction perpendicular to the center axis O of the endoscope 3C.

The operation pad 163A has the switches 162a and 162b corresponding to the up and down directions, and the operation pad 163B has the switches 162c and 162d corresponding to the left and right directions. The other structure is the same as that of the endoscope 3B shown in FIGS. 14A to 14D.

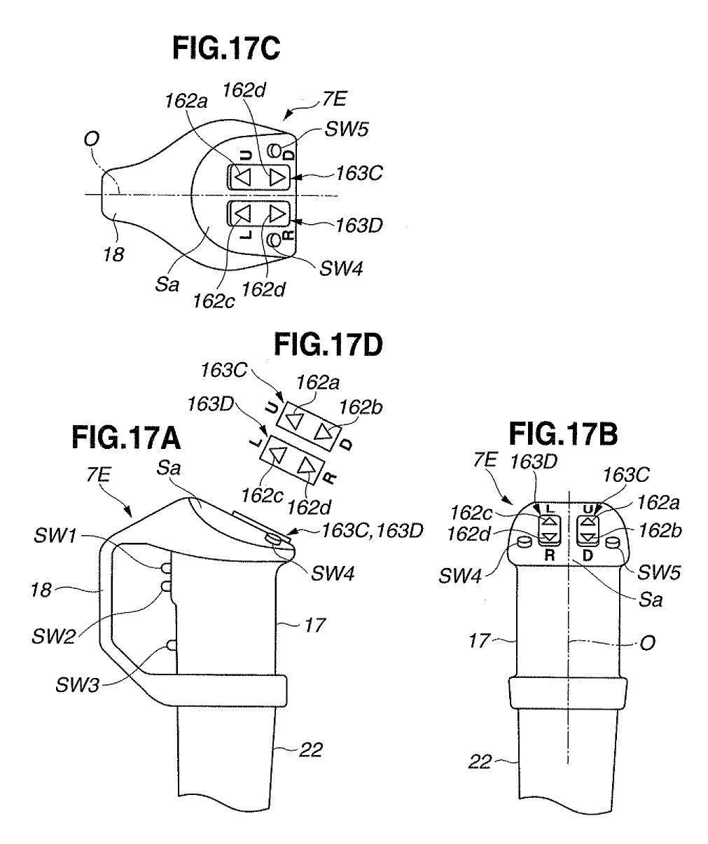

In the endoscope 3D of FIGS. 16A to 16D, the two operation pads 163A and 163B are provided in parallel to the direction perpendicular to the center axis O of the endoscope 3D, but like an endoscope 7E shown in FIGS. 17A to 17D according to a fourth modified example, the two operation pads 163C and 163D may be provided in parallel in a direction parallel to the center axis C of the endoscope 7E.

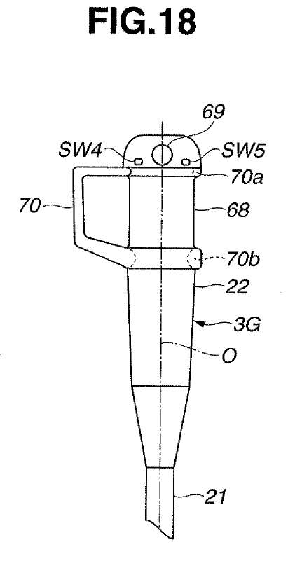

It should be noted that for example, like an endoscope 3G shown in FIG. 18, the hook 70 may be freely turnable with respect to the grasping section 68. The endoscope 3G has ring sections 70a and 70b for allowing the upper end and the lower end of the grasping section 68 of the U-shaped hook 70 in the endoscope 3 shown in FIGS. 8A to 8E, for example, to be freely turnable with respect to the concave portion at the upper end and the lower end of the grasping section 68.

With the above-mentioned structure, the hook 70 can be used while turning if necessary. For example, when the surgeon grasps the grasping section 68 by hand, from the state of FIG. 8A, the hook 70 is turned to a side where the outer side of the grasping part of the hand is covered (the hook is turned to the back side of this sheet of paper in FIG. 8A). In this way, even when the hand grasping the grasping section 68 is in the released state where the hand is not grasping, the endoscope 3G can be held, the operability can be further improved. It should be noted that in the case of the hook 70′ of FIGS. 14A to 14D, the upper end side of he hook 70′ may be set freely turnable.

It should be noted that an embodiment and the like structured by combining parts of the above-mentioned embodiments are also in the scope of the present invention.

1-5. (canceled)

6. An endoscope, comprising:
an elongated insert section having a freely bendable bending section;
an operation section provided on a base end side of the insert section;
a bending instruction input section provided to the operation section;
a grasping section provided on a base end side of the operation section; and
a protruding section protruding in a direction substantially perpendicular to an axis of the grasping section on a base end side of the grasping section.

7. The endoscope according to claim 6, wherein the protruding section has substantially L-shaped section extending from a direction substantially perpendicular to the axis of the grasping section to the insert section.

8. The endoscope according to claim 6, wherein the insert section has a duct line inserted therethrough for allowing a fluid to pass, and a base end side of the duct line penetrates through a tube unit extending from a position nearer to the insert section than the grasping section.

9. The endoscope according to claim 6, wherein:
the grasping section is formed to be substantially bilaterally symmetric to a reference line along a longitudinal direction of the grasping section; and
the endoscope further comprises a plurality of instruction input sections that include the bending instruction input section and are arranged substantially bilaterally symmetric to the reference line along the longitudinal direction of the grasping section.
